(19) United States
(12) Patent Application Publication
(10) Pub. No.: US 2006/0287231 A1
(43) Pub. Date: Dec. 21, 2006

(54) Therapeutic uses of kallikreins

(75) Inventors: Chee-Wui Chu, Montreal (CA); ZhongQi Shao, Montreal (CA); Quan Yang, Montreal (CA); Genevieve Huppe, Montreal (CA)

Correspondence Address: FOLEY AND LARDNER LLP;SUITE 500, 3000 K STREET NW, WASHINGTON, DC, 20007, US

(73) Assignee: IBEX Pharmaceuticals, Inc.

(21) Appl. No.: 11/436,059

(22) Filed: May 18, 2006

Related U.S. Application Data
(60) Provisional application No. 60/681,965, filed on May 18, 2005.

(51) Int. Cl. A61K 38/17

(52) U.S. Cl. 514/12

(57) ABSTRACT

The present invention relates to therapeutic applications of kallikreins and compositions containing them for treating, preventing and/or ameliorating cancers, especially epithelial cancers, such as ovarian, breast and cervical cancer.

### CROSS-REFERENCE TO RELATED PATENT APPLICATIONS

This application claims priority to U.S. provisional application No. 60/681,965, which is incorporated by reference.

### BACKGROUND OF THE INVENTION

The present invention relates to therapeutic applications of kallikreins and compositions containing them for treating, preventing and/or ameliorating cancers, especially epithelial cancers, such as ovarian, breast and cervical cancer.

Cancer is responsible for the majority of deaths both in North America and worldwide. Although the advent of immunobiological approaches and improved chemotherapeutic regimens has increased patient survival once cancer is diagnosed, it has not reduced the overall incidence or severity of the disease.

The treatment of epithelial cancers, such as ovarian, breast and cervical cancer presents significant clinical challenges. Since most ovarian cancer patients are asymptomatic until the disease has metastasized, two thirds are diagnosed with advanced disease. In North America, around 25,000 new cases of ovarian cancer, and about 15,000 deaths from the disease are expected for the year 2006, giving it the highest mortality rate of all gynecological malignancies. Breast cancer is also a significant health problem for women throughout the world. Although important advances have been made in detection and treatment of this disease, breast cancer remains the second leading cause of cancer-related deaths in women, affecting more than 180,000 women in North America each year.

No universally successful method for the prevention or treatment of such epithelial cancers is currently available. Management of these cancers currently relies on a combination of early diagnosis, and aggressive treatment, which may include one or more of a variety of treatments such as surgery, radiotherapy, chemotherapy and hormone therapy. The high mortality observed in ovarian and breast cancer patients indicates that improvements are needed in the treatment of such cancers.

Extracellular proteases have been implicated in the growth, spread and metastatic progression of many cancers and are candidate markers of neoplastic development. This is, in part, due to the ability of malignant cells to dissociate from the primary tumor and to invade new tissues. In order for malignant cells to grow, spread or metastasize, they must have the capacity to invade local host tissue, dissociate or shed from the primary tumor, enter and survive in the bloodstream, invade the new target organ and establish an environment conducive for the new colony growth. During this progression, natural tissue barriers including collagen, laminin, proteoglycans and extracellular matrix glycoproteins such as fibronectin must be degraded in a process brought about by the action of extracellular protease.

Kallikreins are a group of extracellular serine proteases with a molecular weight of 25,000-40,000. The kallikreins have diverse physiological functions in many tissues. Currently, 15 human kallikrein genes have been isolated, characterized, and closely linked on chromosome 19q13.4. The amino acid and nucleotide sequences of all 15 kallikreins are known and accessible in the public databases, such as GenBank. The kallikrein proteins are denoted by the symbol hK and the nucleotides by the symbol KLK.

The amino acid and nucleotide sequences of human kallikrein 5 are accessible in public databases by the accession numbers NP_{—}036559.1 and NM_{—}012427.3. The KLK5 nucleotide sequence is depicted in SEQ.ID.NO:1 and the hK5 amino acid sequence is depicted in SEQ.ID.NO:2.

The amino acid and nucleotide sequences of human kallikrein 6 are accessible in public data bases by the accession numbers AAD51475 and AF149289.1. The KLK6 nucleotide sequence is depicted in SEQ.ID.NO:3 and the hK6 amino acid sequence is depicted in SEQ.ID.NO:4.

The amino acid and nucleotide sequences of human kallikrein 10 are accessible in public data bases by the accession numbers NP_{—}002767 and NM_{—}002776. The KLK10 nucleotide sequence is depicted in SEQ.ID.NO:5 and the hK10 amino acid sequence is depicted in SEQ.ID.NO:6.

The amino acid and nucleotide sequences of human kallikrein 13 are accessible in public data bases by the accession numbers NP_{—}056411.1 and NM_{—}015596.1. The KLK13 nucleotide sequence is depicted in SEQ.ID.NO:7 and the hK13 amino acid sequence is depicted in SEQ.ID.NO:8.

In recent years, there have been numerous scientific papers relating to the association between human kallikreins and various types of cancers. Much of the evidence suggests that kallikreins, with the exception of kallikreins 2 and 10, are associated with malignancy, and cause tumor growth, shedding of tumor cells and invasion of target organs.

There is a need for additional treatment options for cancer patients.

### SUMMARY OF THE INVENTION

In one embodiment, the current invention is a method of treating cancer in a subject afflicted therewith, which comprises contacting the cancer cells in said subject with an effective amount of a composition selected from the group consisting of: a specific individual human kallikrein, other than kallikreins 2 and 10; and a plurality of human kallikreins. The cancer may be selected from the group consisting of ovarian, breast, or cervical cancer.

In a further embodiment, the kallikrein bioactivity associated with the cancer cells is enhanced by direct administration of the specific individual kallikrein. The individual kallikrein may be selected from the group consisting of kallikrein 5 and kallikrein 13.

In a further embodiment of the current invention, the kallikrein bioactivity associated with the cancer may also be enhanced by direct administration of a combination of kallikreins. In another embodiment the combination of kallikreins is selected from the group consisting of kallikreins 5 and 6; kallikreins 5 and 10; kallikreins 5, 6 and 10; kallikreins 5, 6 and 13; and kallikreins 5, 10 and 13. In a further embodiment, the combination includes kallikrein 5.

In another embodiment of the current invention, the combination of kallikreins exhibit synergistic activity in inhibiting the growth of cancer cells. Such a combination of kallikreins may comprises hK5 and hK10.

In a further embodiment, the kallikrein bioactivity is enhanced by direct administration of a modulator for the kallikreins. The modulator may be selected from the group consisting of a protein, a nucleic acid, a, hormone, a naturally occurring cognate ligand of the kallikrein, a peptidomimetic or a small molecule.

In a further embodiment, the kallikrein bioactivity is enhanced by introducing into the cancer cells a recombinant expression vector comprising a plurality of nucleotide sequences encoding the corresponding kallikreins and which can be induced to express said kallikreins. The expression vector may be selected from the group consisting of retroviruses, adenovirus, herpes and vaccinia vectors, containing appropriate regulatory elements.

In a further embodiment, the kallikrein bioactivity is enhanced by direct introduction into the cancer cells or surrounding tissue of a plurality of naked polynucleotides operatively coding for the corresponding kallikreins. Alternatively, the kallikrein bioactivity is enhanced by introducing into the subject afflicted by cancer, tumor cells, stably transfected with a recombinant expression vector comprising a plurality of nucleotide sequences coding for the corresponding kallikreins.

Another embodiment of the invention is a pharmaceutical composition for treating cancer in which the composition is selected from the group consisting of:

a specific kallikrein, other than kallikreins 2 and 10:

a plurality of kallikreins; and

a kallikrein modulator,

in association with a pharmaceutically acceptable carrier.

Another embodiment of the invention is a pharmaceutical composition comprising kallikreins 5 and 10 in association with a pharmaceutically acceptable carrier.

Another embodiment of the invention is a pharmaceutical composition comprising a plurality of naked polynucleotides operatively coding for the corresponding kallikreins in association with a pharmaceutically acceptable carrier.

Another embodiment of the invention is a recombinant expression vector comprising a plurality of nucleotide sequences coding for the corresponding kallikreins. A tumor cell containing such a recombinant expression vector is also within the scope of the invention.

### BRIEF DESCRIPTION OF THE DRAWINGS

FIG. 1 shows the effect of expression of combinations of kallikreins on invasiveness of ES-2 ovarian cancer cells;

FIG. 2 shows the results of survival of mice injected with control (transfected with an empty vector) ovarian cancer cell lines and 4 groups of treated (transfected with kallikrein expression vectors) ovarian cancer cell lines where the treated groups overexpress one or more kallikreins;

FIG. 3 shows the results of survival of mice injected with control (transfected with an empty vector) ovarian cancer cell line and/or mice injected with treated ovarian cancer cell line (transfected with hK10 expression vector) expressing hK10 at a low level;

FIG. 4 shows the synergistic effect of expressing very low levels of hK5 and hK10 on the survival of mice injected with ES-2 cells transfected with expression vectors carrying KLK5 and KLK10.

### DETAILED DESCRIPTION OF THE INVENTION

This invention is based on the discovery that specific individual kallikreins (other than kallikreins 2 and 10) can act as tumor suppressors, and also that a combination of two or more kallikreins, in which the component kallikreins are present in amounts that do not show tumor suppressor activity individually, also exhibit such activity. On account of their tumor suppressor properties, both the individual kallikreins and the combinations of kallikreins are useful in the treatment of cancers, particularly epithelial cancers, such as ovarian, breast and cervical cancer.

An object of this invention is to provide improved methods for the treatment of cancers, particularly epithelial cancers such as ovarian, breast and cervical cancer.

Another object of this invention is to provide pharmaceutical compositions for use in the improved methods, which compositions contain kallikreins or kallikrein modulators as the active agent.

Broadly, the invention provides a method of treating a subject afflicted with cancer, which involves contacting the cancer cells of the subject with an effective amount of a specific individual human kallikrein, other than kallikreins 2 and 10, or a plurality (2, 3 or more) of human kallikreins.

“Contacting” in relation to the cancer cells may result from kallikreins within the cancer cell, secreted by the cell and/or present in the extracellular milieu surrounding the cell. Enhancing the bioactivity and/or expression of kallikreins in and/or around the cancer cells inhibits the development of cancer.

The term “treating a subject afflicted with cancer” as used herein, is meant the inhibition of the growth and spread of cancer cells in the subject. Preferably, such treatment also leads to the regression of tumor growth, i.e. a decrease in size of a measurable tumor.

The term “cancer” as used herein, encompasses a wide range of malignant tumors that are capable of invasive growth and spread (metastasis) throughout the body via the lymphatic system and/or the blood stream. Preferably, the cancer is an epithelial cancer, more particularly ovarian, breast, or cervical cancer.

The term “effective amount” as used herein, means a course of therapy which will result in treating cancers, particularly epithelial cancers. It will be appreciated that the actual preferred course of therapy will vary according to inter alia, the mode of administration, the particular formulation of the kallikrein(s) and the individual subject being treated. The optimum course of therapy for a given set of conditions can be readily ascertained by those skilled in the art.

In another embodiment the invention features the use of kallikrein modulators to enhance and/or prolong the activity of endogenous kallikreins or to increase the expression of endogenous kallikreins In further embodiments, the invention features pharmaceutical compositions containing specific individual kallikreins, other than kallikreins 2 and 10, combinations of kallikreins, kallikrein modulators, expression vectors cells comprising a plurality of polynucleotides that encode for the corresponding kallikreins.

Unless otherwise defined herein, scientific and technical terms used in connection with the present invention shall have the meanings that are commonly understood by those of ordinary skill in the art, to which this invention belongs. Further, unless otherwise required by context, singular terms shall include pluralities and plural terms shall include the singular unless the context otherwise requires. In general, the technical terms and procedures used herein are conventional cell and tissue culture, molecular biology and microbiology terms and procedures within the knowledge of those skilled in the art. Such terms and techniques are explained fully in the literature. See, e.g. Sambrook and Russell “Molecular Cloning: A Laboratory Manual” Third Edition (2001) Cold Spring Harbor Laboratory Press, Cold Spring Harbor, N.Y.; D. M. Glover and B. D. Hames “DNA Cloning: A Practical Approach” Volumes I and II (2002); Oxford University Press, Oxford; Ausubel et. al. “Current Protocols in Molecular Biology” (2005) Wiley-Liss, Hoboken, N.J. B. D. Hames & S. J. Higgins eds. “Nucleic Acid Hybridization” (1985); B. D. Hames & S. J. Higgins eds. “Transcription and Translation” (1984); R. I. Freshney “Culture of Animal Cells: A Manual of Basic Techniques” Fourth Edition (2005), Wiley-Liss, Hoboken, N.J.; Martin Tymms “In Vitro Transcription and Translation: Methods in Molecular Biology” (2003); B. Perbal, “A Practical Guide to Molecular Cloning” (1984); Alan Rolland ed “Advanced Gene Delivery: From Concepts to Pharmaceutical Products” (2005); Harwood Academic Publishers, Amsterdam, The Netherlands; K. Taira et. al. “Non-Viral Gene Therapy: Gene Design and Delivery” (2005) Springer.

In one embodiment, enhancement of the kallikrein bioactivity in or around the cancer cells results from the direct administration to the subject of a specific individual human kallikrein, other than kallikrein 2 and 10, or a plurality of human kallikreins. Preferably the kallikreins are used in the form of proteins.

Preferred, individual kallikreins are kallikrein 5 and kallikrein 13. In the combinations the preferred kallikreins are kallikreins 5, 6, 10 and 13. Desirably, kallikrein 5 is a component of any combination. Illustrative combinations are kallikreins 5 and 6; kallikreins 5 and 10; kallikreins 5, 6 and 10; kallikreins 5, 6 and 13 and kallikreins 5, 10 and 13.

Quite surprisingly certain combinations of the kallikreins exhibit synergistic effects in inhibiting the growth of cancer cells. Illustrative of such combinations are those comprising hK5 and hK10, as shown in Example 6 herein.

Another embodiment of the invention provides an alternative approach to the enhancement or prolongation of the kallikrein bioactivity in or around the cancer cells, in which a kallikrein modulator is directly administered to the subject.

The term “modulator” as used herein, refers to compounds that affect the activity of the kallikreins in vivo. Modulators can be agonists or other substances that exert their effect on kallikrein activity via enzymatic activity, expression, post translational modifications or by other means. Modulators of the kallikreins are molecules which, enhance or prolong their activity. Modulators of the kallikreins include proteins, nucleic acids, hormones, carbohydrates, peptidomimetics or small molecules (less than approximately 500 Dalton) or any other molecule which activates kallikreins.

Suitable modulators may be identified by standard screening assays in which compounds or agents which bind to the kallikrein and/or have a stimulatory effect on the activity or the expression of the kallikrein. These assays may employ cells which express the particular kallikreins(s), so-called cell-based assays. Alternatively, the assays may be based on the isolated kallikreins, so-called cell-free assays. As is well known by those skilled in the art such assays may be adapted to high-throughput screening of chemical libraries, making them particularly suitable for identifying small molecule drug candidates. The assays can be performed in a variety of formats, including protein-protein binding assays, biochemical screening assays, immunoassays and cell-based assays which are well characterized in the art.

In another embodiment of the invention, a similar result to that described above may be achieved by administering promoters that enhance expression of endogenous kallikreins.

The kallikreins, singly or in combination, or the kallikrein modulators are preferably administered to the subject in isolated and purified forms, desirably in association with a pharmaceutically acceptable carrier. The preferred route of administration for the kallikreins is parenteral including intravenous, intramuscular, intraperitoneal or subcutaneous injection. Alternately the kallikrein can be delivered by injection directly into the tumor site or implantation of a device containing a slow release formulation. The kallikrein(s) composition can also be incorporated into liposomes or other carrier vehicles to facilitate delivery to the cancer cells or tumor site. The modulators may be delivered by oral or parenteral routes of administration.

The compositions described herein can be prepared by known methods for the preparation of pharmaceutically acceptable compositions which can be administered to subjects, such that an effective quantity of the kallikrein or modulator is combined in a mixture with a pharmaceutical acceptable carrier. Suitable carriers are described for example in Remington's Pharmaceutical Sciences (Mack Publishing Company, Easton, Pa. USA (1985). Illustrative of carriers suitable for parenteral administration include: aqueous solutions, physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the kallikrein(s) may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Pharmaceutical compositions suitable for use in the invention include compositions in which the kallikreins, singly or in combination, or modulators thereof, are present in an effective amount to treat the cancer. The determination of an effective dose is well within the capability of those skilled in the art. Normal dosage amounts may vary from 0.001 to 1,000 mg/kg/day.

Variants of kallikreins, including substitution, addition, and deletion variants of kallikreins, may be used according to the invention. Such variants may be conservatively substituted variants which differ only by conservative amino acid substitutions, for example, substitution of one amino acid for another of the same class (e.g. valine for glycine or arginine for lysine), or by one or more non-conservative substitutions, deletions, or insertions located at positions of the amino acid sequence which do not destroy the function of the kallikrein as measured by appropriate assays.

Preferably such a variant sequence is at least 85% more preferably 90%, and most preferably 95% identical at the amino acid level to the sequence of the known kallikrein.

In yet another embodiment the invention provides indirect methods for enhancing kallikrein bioactivity in or around the cancer cells.

In one such method, a nucleotide sequence encoding the corresponding kallikrein or a plurality of nucleotide sequences encoding the corresponding kallikreins may be used to enhance kallikrein expression.

Recombinant expression vectors derived from retroviruses, adenovirus, herpes or vaccinia viruses may be used for delivery of the nucleotide sequences to the targeted organ, tissue or cell population. In addition to the nucleotide sequences such expression vectors contain the usual regulatory elements such as promoters. Methods which are well known to those skilled in the art can be used to construct recombinant vectors which will express kallikreins, corresponding to the nucleotides of the genes encoding them. These methods are described both in Sambrook et. al. (supra) and in Ausubel et. al. (supra).

Genes encoding the kallikreins can be turned on by transforming a cell or tissue with expression vectors which express high levels of a nucleotide operatively coding for the corresponding kallikrein protein. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

Many methods for introducing vectors into cells or tissues are available and equally suitable for use in vivo, in vitro, and ex vivo. For ex vivo therapy, vectors may be introduced into tumor cells taken from the patient and clonally propagated for autologous transplant back into that same patient. Delivery by transfection, by liposome injections, or by polycationic amino polymers may be achieved using methods which are well known in the art. (See, e.g. Goldman, C. K. et. al (1997) Nat. Biotechnol 15:462-466).

An alternative indirect method suitable for the present invention utilizes ex vivo transfection of cells removed from a cancer with nucleotide sequences encoding specific kallikreins or plurality of kallikreins to obtain high expression levels and injecting the transfected cells back into the cancer.

Yet another such method involves the direct introduction into the cancer cells or a tumor containing the cells, of a “naked” polynucleotide or polynucleotides operatively coding for the corresponding kallikrein or combination of kallikreins. Directly introduction of the “naked” polynucleotide may be accomplished by any of several procedures, all of which are more fully described in Rolland (supra) and Taira (supra).

The “subject” treated according to the present invention may be any animal afflicted by cancer or a tumor. Typically, the subject is a mammal, for instance a human.

The following examples are offered for illustrative purposes only and are not intended to limit the scope of the present invention in any way. All patents and literature references cited in the present specification are hereby incorporated by reference in their entirety. Commercially available reagents referred to in the examples were used according to the manufacturer's instructions unless otherwise indicated.

### EXAMPLE 1

### Correlation Between Kallikrein Expression by Ovarian Cancer Cell Lines and Tumorigenic Phenotype

Human ovarian cancer cell lines (listed in Table 1) were tested for kallikrein expression by specific ELISAs. Culture media were harvested when cells reached confluency and tested for secreted hK5, hK6, hK10 and hK13. It had been determined in previous experiments that kallikreins are very efficiently secreted by human ovarian cancer cell lines and mostly found in the culture medium. These cell lines were also tested in the in vitro Biocoat Tumor Invasion System invasion assay (BD Biosciences, www.bdbiosciences.com) and in soft agar anchorage independence assay. The Biocoat Tumor Invasion System was use as recommended by the manufacturer. In the anchorage independence assay, cells were suspended in 0.35% agarose at 1600 cells/mL. 3 mL of cell suspension was plated into a well of a 6 well culture plate pre-coated with 2 mL of 0.7% agarose. The soft agar was overlaid with 0.5 mL of medium which was replaced every 3 days. Colony formation was determined at 10 days. All cells were grown in DMEM (Dulbeco's Minimum Essential Medium) supplemented with 10% fetal bovine serum. All cultures were incubated at 37° C. with 5% CO₂.

Additionally, the tumorigenicity of each cell line in nude mice was also determined. The respective cells were injected into the peritoneal cavity of nude mice at 1×10⁷ cells/mouse in 1 mL PBS. The ability of cells to form tumors and the survival of the host animals were determined.

TABLE 1
Correlation between kallikrein expression by ovarian cancer cell
lines and tumorigenic phenotype

| | | | | Xenograft | |
|---|---|---|---|---|---|
| | | | | (in mice) | |
| | hK | In vitro | Anchorage | Form | Survival |
| Cell Line | Expression | Invasion | Independence | tumor | (days) |
| CaOv-3 | + | − | − | ND | ND |
| OvCaR-3 | + | − | − | 0/6 | NA |
| OvCaR-4 | + | − | − | ND | ND |
| OV2008 | + | − | − | 4/5 | 66 days |
| C13 | + | − | − | 0/5 | NA |
| Ov-90 | + | − | − | ND | ND |
| OvCa433 | + | − | − | 0/3 | NA |
| Sk-Ov-3 | +/− | +/− | +/− | 2/7 | 105 |
| OvCa429 | − | + | + | 3/3 | 62 |
| Hey | − | + | + | 2/3 | 24 |
| ES-2 | − | + | + | 5/5 | 16 |
| OCC-1 | − | − | + | 3/3 | 15 |
| A2780cp | − | − | + | 3/3 | 24 |
| A2780s | − | − | + | 3/3 | 46 |

ND—not determined
NA—not applicable because cells did not form tumor and survival of mice was not affected.

Ovarian cancer cell lines that expressed kallikreins, expressed multiple kallikreins. All of the hK expressing cell lines did not grow in soft agar and were less invasive in the Biocoat Tumor Invasion System assay. Additionally they were less aggressive in nude mice, three failing to form tumors. Three of the six cell lines that did not express kallikreins were invasive and all six grew in soft agar. Additionally, all six were more aggressive in mice, forming tumors and resulting in significantly shorter survival.

### EXAMPLE 2

### Construction of Mammalian Expression Vectors and Transfection of ES-2 Cells (ATCC® #CRL-1978)

Construction of Expression Vector.

The coding region of the appropriate kallikrein gene (KLK5, 6, 10 or 13) was amplified from the respective cDNA. Amplified DNA was digested with the appropriate restriction enzymes and inserted into the expression vector. As an example, the following protocol was used to construct a hK5 expression vector. KLK5 cDNA on pOTB7 was obtained from the ATCC. Oligos, KLK5F1 (GCTCTAGAATGGCTACAGCAAGACC) (SEQ ID No: 9) and KLK5R1 (TATCCGGAGTCTGAAAGGAGTGTCAG) (SEQ ID No: 10), were used to amplify the coding region for hK5 protein by PCR from plasmid pOTB7-KLK5 (ATCC). Amplified DNA fragment was digested with XbaI/BspEI and inserted into pIRESpuro-2 (Clontech, clontech.com), pre-digested with NheI/BspEI.

Protocol for Cell Transfection.

ES-2 cells were transfected with expression vectors using Lipofectomine™ 2000 (Invitrogen, www.invitrogen.com). Transfection procedure was as recommended by manufacturer.

ES-2 cells expressing 2 kallikreins were transfected sequentially. As an example, ES-2 cells stably transfected with KLK10 in the pCMV-neo vector were then transfected with KLK5 in the pIRESpuro-2 vector.

### EXAMPLE 3

### Synergistic Effect of hK5 in Combination with Another Kallikrein on the Invasiveness of Ovarian Cancer Cells

The following transfected cell lines were tested in the Biocoat Tumor Invasion assay:

ES-2: untransfected ovarian cancer cell line.

ES2-DTC-2: ES-2 cells transfected with control vector, no kallikrein expression.

ES2-hK5/6-1: ES-2 cells transfected with KLK5 and 6, expressing both.

ES2-hK5/6-2: ES-2 cells transfected with KLK5 and 6, lost hK5 expression.

ES2-hK5/10-B1: ES-2 cells transfected with KLK5 and 10, lost hK5 expression.

ES2-hK5/10-B2: ES-2 cells transfected with KLK5 and 10, expressing both.

ES2-hK6/10-2: ES-2 cells transfected with KLK6 and 10, expressing both.

Results:

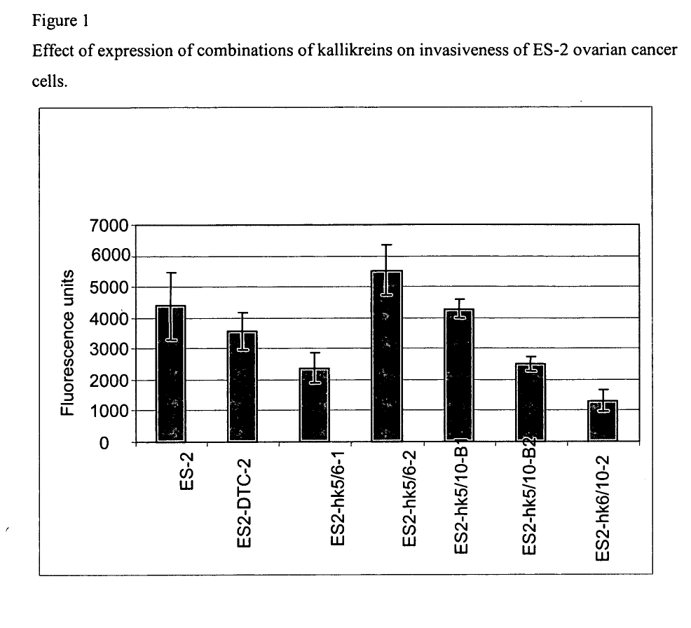

The Biocoat Tumor Invasion results are shown in FIG. 1, wherein higher fluorescence units is equivalent to greater invasion. ES-2 cells doubly transfected with kallikreins 5 and 6, kallikreins 5 and 10 or kallikreins 6 and 10 had lower invasion than control untransfected ES-2 cells. The doubly transfected cells that had lost expression of hK5 (ES2-hK5/6-2 and ES2-hK5/10-B1) did not show significant reduction in invasion.

### EXAMPLE 4

### Synergistic Effect of Human Kallikreins on Angiogenesis

A human umbilical vascular endothelial cell (HUVEC) capillary formation assay was used to assess the anti-angiogenic effect of 4 human kallikreins (hK5, 6, 10 and 13) in various combinations. These recombinant human kallikreins were produced in Pichia pasturis or mammalian cells and purified to >95% purity. The kallikreins were used at sub-optimal doses that did not have an effect on capillary formation individually. 96 well culture plate was coated with Matrigel (BD Biosciences) at 50 μL/well. After the gel had set, 50 μL of EGM-2 medium (Cambrex, www.cambrex.com) containing the test kallikrein was added to each well, then 5000 HUVEC cells (Cambrex) were added to each well in another 50 μL of medium. After 24 hours at 37° C., the cells were stained with Calcein AM (Invitrogen). Image of the well was captured with a fluorescent microscope and capillary formation was determined with NIH Image software.

TABLE 2

Combination of
Human Kallikreins Tested

| hK5 | hK6 | hK10 | hK13 | Anti-angiogenic |
|---|---|---|---|---|
| (20 nM) | (20 nM) | (1 μM) | (20 nM) | effect |
| + | + | | | − |
| + | | + | | − |
| + | | | + | − |
| | + | | + | − |
| | + | + | | − |
| | | + | + | − |
| + | + | + | | + |
| + | + | | + | + |
| + | | + | + | + |
| | + | + | + | − |

Results

At sub-optimal doses, the four human kallikreins tested showed a synergistic effect in suppressing the formation of capillaries by HUVEC cells. Any combination of three of the four kallikreins which included hK5 was effective.

### EXAMPLE 5

### Effect of Expression of Kallikreins by ES-2 Ovarian Cancer Cells on Survival Time in a Nude Mouse Xenograft Model

Cell Culture Conditions

Cells were cultured at 37° C., 5% CO₂, in DMEM containing 2.5% fetal bovine serum, and 7.5% normal bovine serum and penicillin/streptomycin.

Description of Xenografted Cells

ES-2 cells were transfected as described above.

The study compared 5 stably transfected ES-2 ovarian cancer cells and the parental untransfected ES-2 cell line as follows:

1: control group 1—ES-2 ovarian cancer cells

2: control group 2—stable transfected ES-2 cells expressing no kallikreins

3: ES-2 cells stably transfected and overexpressing hK5

4: ES-2 cells stably transfected and overexpressing hK6

5: ES-2 cells stably transfected and overexpressing hK10

6: ES-2 cells stably transfected and overexpressing hK5 and hK6

Animal Tumour Model

Female CD-1 nu/nu mice (5 weeks old) were acclimatised to their environment for seven days prior to the initiation of the study. The animals were randomly divided into 6 groups (8 animals/group). Groups were blinded to prevent bias in endpoint assessment. Cultured test cells (as listed above) were harvested from monolayer cultures and resuspended in PBS for injection intra-peritoneally (i.p.) into each mouse (1×10⁷ cells, total volume 1 ml). Each group was injected with one of the 6 transfected cell lines listed above. Study duration was 8 weeks. The animals were monitored daily for general wellness and body weight. Animals still alive at the end of treatment were sacrificed. Ascites fluids were collected at necropsy.

Results

Culture medium harvested from the respective cell lines and the ascites fluids collected at necropsy were tested for kallikreins. This confirmed that the cell lines were expressing the appropriate kallikreins and continue to express the same kallikreins in the host animals.

| Sample | | | |
|---|---|---|---|
| Culture media | hK5 (ng/mL) | hK6 (ng/mL) | hK10 (ng/mL) |
| 1 (ES-2) | 0 | 0 | 0 |
| 2 (ES-2 vector | 0 | 0 | 0 |
| control) | | | |
| 3 (ES-2/hK5) | 227 | 0 | 0 |
| 4 (ES-2/hK6) | 0 | 351 | 0 |
| 5 (ES-2/hK10) | 0 | 0 | 316 |
| 6 (ES2/hK5 + 6) | 58 | 202 | 0 |

| Ascites fluids | | | |
|---|---|---|---|
| from group | Average concentration | | |
| 1 (ES-2) | 0 | 0 | 0 |
| 2 (ES-2 vector | 0 | 0 | 0 |
| control) | | | |
| 3 (ES-2/hK5) | 1000 | 0 | 0 |
| 4 (ES-2/hK6) | 0 | 522 | 0 |
| 5 (ES-2/hK10) | 0 | 0 | 3218 |
| 6 (ES2/hK5 + 6) | 2000 | 1200 | 0 |

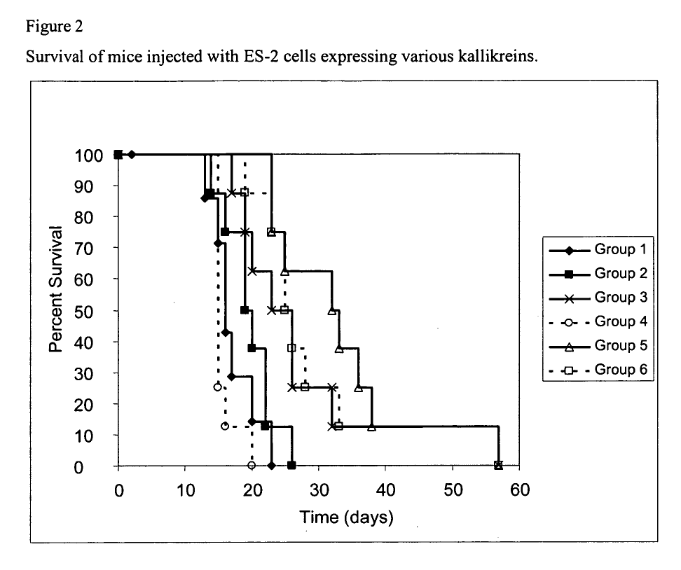

Groups 3, 5 and 6 showed increased survival compared to control groups 1 and 2 as shown in FIG. 2. Thus the expression of hK5, hK10 or hK5 in combination with hK6 prolonged the survival of the test animals.

### EXAMPLE 6

### Synergistic Effect of Expression of Multiple Kallikreins by ES-2 Ovarian Cancer Cells on Survival Time in Nude Mouse Xenograft Model

Description of Xenografted Cells

The study compared 4 stably transfected ES-2 cell lines as follows:

1: Control group for ES-2 cells with single vector (control 1)

2: ES-2 cells stably over-expressing hK10 (ES-2/hK10)

3: Control group for ES-2 cells with two vectors (control 2)

4: ES-2 cells stably over-expressing hK5 and hK10 (ES-2/hK5+10)

Cell Culture Conditions

Cells were cultured at 37° C., 5% CO₂, in DMEM containing 2.5% fetal bovine serum, and 7.5% normal bovine serum and penicillin/streptomycin.

Animal Tumour Model

Female CD-1 nu/nu mice (5 weeks old) were acclimatised to their environment for seven days prior to the initiation of the study. The animals were randomly divided into 4 groups (8 animals/group). Groups were blinded to prevent bias in endpoint assessment. Cultured test cells (as listed above) were harvested from monolayer cultures and resuspended in PBS for injection intra-peritoneally (i.p.) into each mouse (1×10⁷ cells, total volume 1 ml). Each group was injected with one of the 4 transfected cell lines listed above. Study duration was 8 weeks. The animals were monitored daily for general wellness and body weight. Animals still alive at the end of treatment were sacrificed. Ascites fluids were collected at necropsy.

Results

Culture medium harvested from the respective cell lines and the ascites fluids collected at necropsy were tested for kallikreins. This confirmed that the cell lines were expressing the appropriate kallikreins and continue to express the same kallikreins in the host animals. This also established the expression levels of each cell line.

| Sample | | |
|---|---|---|
| Culture media | hK5 (ng/mL) | hK10 (ng/mL) |
| 1 (control 1) | 0 | 0 |
| 2 (ES-2/hK10) | 0 | 150 |
| 3 (control 2) | 0 | 0 |
| 4 (ES-2/hK5 + 10) | 74 | 40 |

| | |
|---|---|
| Ascites fluids from group | Average concentration |

| | | |
|---|---|---|
| 1 (control 1) | 0 | 0 |
| 2 (ES-2/hK10) | 0 | 1257 |
| 3 (control 2) | 0 | 0 |
| 4 (ES-2/hK5 + 10) | 393 | 464 |

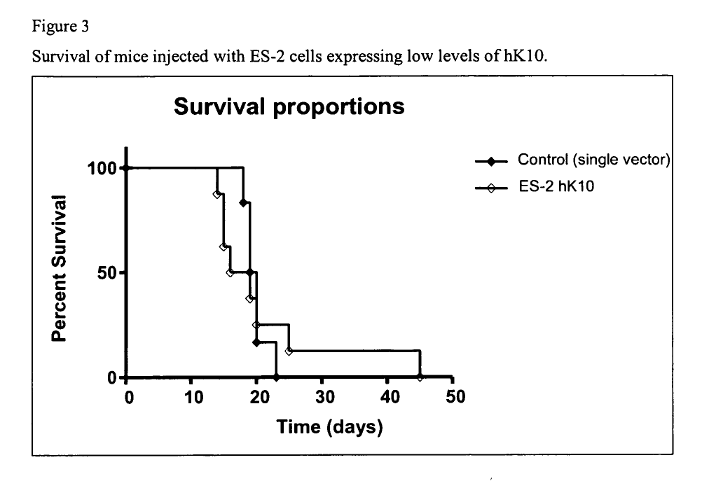

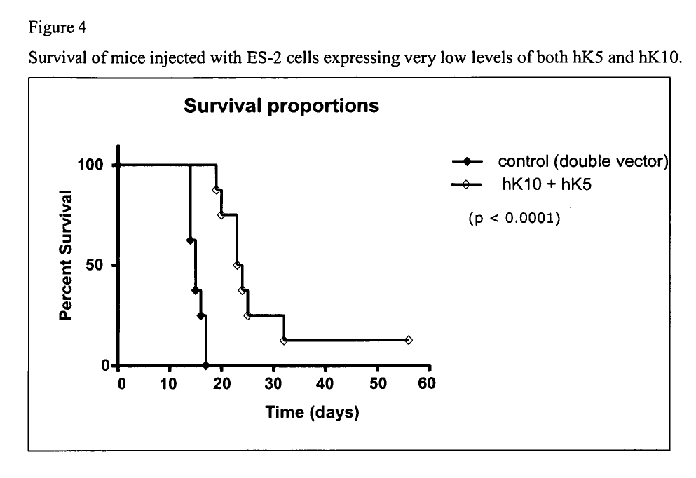

ES-2 cells expressing only hK10 (ES-2/hK10) at approximately half the level of cells in the previous animal study (example 5) did not show increased survival compared to the vector control cell line (control 1) (FIG. 3). ES-2 cells expressing hK5 and hK10 (ES-2/hK5+10) at very low levels showed a significant increase in survival compared to the vector control cell line (control 2) (FIG. 4). This demonstrates that ES-2 cells expressing a combination of hK5 and hK10 at approximately 8 fold lower levels had similar survival advantage as the ES-2 cells expressing hK10 alone.

It will occur to those of ordinary skill in the art that various modifications may be made to the disclosed embodiments and that such modifications are intended to be within the scope of the present invention.

What is claimed is:

1. A method of treating cancer in a subject afflicted therewith, which comprises contacting the cancer cells in said subject with an effective amount of a composition selected from the group consisting of:
a specific individual human kallikrein, other than kallikreins 2 and 10; and
a plurality of human kallikreins.

2. A method as claimed in claim 1, in which kallikrein bioactivity associated with the cancer cells is enhanced.

3. A method as claimed in claim 2, in which kallikrein bioactivity is enhanced by direct administration of the specific individual kallikrein.

4. A method as claimed in claim 3, in which the individual kallikrein is selected from the group consisting of kallikrein 5 and kallikrein 13.

5. A method as claimed in claim 2, in which kallikrein bioactivity is enhanced by direct administration of a combination of kallikreins.

6. A method as claimed in claim 5, in which the combination of kallikreins is selected from the group consisting of kallikreins 5 and 6; kallikreins 5 and 10; kallikreins 5, 6 and 10; kallikreins 5, 6 and 13; and kallikreins 5, 10 and 13.

7. A method as claimed in claim 6, in which the combination includes kallikrein 5.

8. A method as claimed in claim 6, in which the combination of kallikreins exhibit synergistic activity in inhibiting the growth of cancer cells.

9. A method is claimed in claim 8, in which the combination comprises hK5 and hK10.

10. A method as claimed in claim 2, in which kallikrein bioactivity is enhanced by direct administration of a modulator for the kallikreins.

11. A method as claimed in claim 10, in which the modulator is selected from the group consisting of a protein, a nucleic acid, a, hormone, a naturally occurring cognate ligand of the kallikrein, a peptidomimetic and a small molecule.

12. A method as claimed in claim 2 in which kallikrein bioactivity is enhanced by introducing into the cancer cells a recombinant expression vector comprising a plurality of nucleotide sequences encoding the corresponding kallikreins and which can be induced to express said kallikreins.

13. A method as claimed in claim 12, in which the expression vector is selected from the group consisting of retroviruses, adenovirus, herpes and vaccinia vectors, containing appropriate regulatory elements.

14. A method is claimed in claim 2 in which kallikrein bioactivity is enhanced by direct introduction into the cancer cells or surrounding tissue of a plurality of naked polynucleotides operatively coding for the corresponding kallikreins.

15. A method as claimed in claim 2, in which kallikrein bioactivity is enhanced by introducing into the subject afflicted by cancer, tumor cells, stably transfected with a recombinant expression vector comprising a plurality of nucleotide sequences coding for the corresponding kallikreins.

16. A pharmaceutical composition for treating cancer in which the composition is selected from the group consisting of:
a specific kallikrein, other than kallikreins 2 and 10:
a plurality of kallikreins; and
a kallikrein modulator,
in association with a pharmaceutically acceptable carrier.

17. A pharmaceutical composition comprising kallikreins 5 and 10 in association with a pharmaceutically acceptable carrier.

18. A pharmaceutical composition comprising a plurality of naked polynucleotides operatively coding for the corresponding kallikreins in association with a pharmaceutically acceptable carrier.

19. A recombinant expression vector comprising a plurality of nucleotide sequences coding for the corresponding kallikreins.

20. A tumor cell containing the vector of claim 19.

21. The method of claim 1, wherein the cancer is selected from the group consisting of: breast, ovarian, and cervical cancer.


SEQUENCE LISTING (10 sequences)

SEQ ID NO: 1 - DNA, 1528 bp, Homo sapiens
ggaaggcacaggcctgagaagtctgcggctgagctgggagcaaatcccccaccccctacctgggggacagggcaagtgagacctggtgagggtggctcagcaggaagggaaggagaggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacctggggaaataaggtgcagcggccatggctacagcaagacccccctggatgtgggtgctctgtgctctgatcacagccttgcttctgggggtcacagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaaagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaagtggaaaaaaaaaaaaaaaaaaaaaaaaaaaaaaaaaaa

SEQ ID NO: 2 - protein, 293 aa, Homo sapiens
MATARPPWMWVLCALITALLLGVTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 3 - DNA, 1451 bp, Homo sapiens
gtcgacccacgcgtccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaattttattatgaaaaaaaaaaaaaaaaaaaaaaaaaaaagggcggccgc

SEQ ID NO: 4 - protein, 244 aa, Homo sapiens
MKKLMVVLSLIAAAWAEEQNKLVHGGPCDKTSHPYQAALYTSGHLLCGGVLIHPLWVLTAAHCKKPNLQVFLGKHNLRQRESSQEQSSVVRAVIHPDYDAASHDQDIMLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAK

SEQ ID NO: 5 - DNA, 1454 bp, Homo sapiens
accagcggcagaccacaggcagggcagaggcacgtctgggtcccctccctccttcctatcggcgactcccagatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggcgccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgatcacctgctgcttcttcagggcgagcagctccgccggacgactcgctctgttgtccatcccaagtaccaccagggctcaggccccatcctgccaaggcgaacggatgagcacgatctcatgttgctaaagctggccaggcccgtagtgccggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccccgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatgtgattgtcatgtaaggcttaacacagtgggtggtgagttctgactaaaggttacctgttgtcgtgaaaaaaaaaaaaaa

SEQ ID NO: 6 - protein, 276 aa, Homo sapiens
MRAPHLHLSAASGARALAKLLPLLMAQLWAAEAALLPQNDTRLDPEAYGSPCARGSQPWQVSLFNGLSFHCAGVLVDQSWVLTAAHCGNKPLWARVGDDHLLLLQGEQLRRTTRSVVHPKYHQGSGPILPRRTDEHDLMLLKLARPVVLGPRVRALQLPYRCAQPGDQCQVAGWGTTAARRVKYNKGLTCSSITILSPKECEVFYPGVVTNNMICAGLDRGQDPCQSDSGGPLVCDETLQGILSWGVYPCGSAQHPAVYTQICKYMSWINKVIRSN

SEQ ID NO: 7 - DNA, 1258 bp, Homo sapiens
tcaggccccgcccgccctgccctcccctcccgatcccggagccatgtggcccctggccctagtgatcgcctccctgaccttggccttgtcaggaggtgtctcccaggagtcttccaaggttctcaacaccaatgggaccagtgggtttctcccaggtggctacacctgcttcccccactctcagccctggcaggctgccctactagtgcaagggcggctactctgtgggggagtcctggtccaccccaaatgggtcctcactgccgcacactgtctaaaggaggggctcaaagtttacctaggcaagcacgccctagggcgtgtggaagctggtgagcaggtgagggaagttgtccactctatcccccaccctgaataccggagaagccccacccacctgaaccacgaccatgacatcatgcttctggagctgcagtccccggtccagctcacaggctacatccaaaccctgcccctttcccacaacaaccgcctaacccctggcaccacctgtcgggtgtctggctggggcaccaccaccagcccccaggtgaattaccccaaaactctacaatgtgccaacatccaacttcgctcagatgaggagtgtcgtcaagtctacccaggaaagatcactgacaacatgttgtgtgccggcacaaaagagggtggcaaagactcctgtgagggtgactctgggggccccctggtctgtaacagaacactgtatggcatcgtctcctggggagacttcccatgtgggcaacctgaccggcctggtgtctacacccgtgtctcaagatacgtcctgtggatccgtgaaacaatccgaaaatatgaaacccagcagcaaaaatggttgaagggcccacaataaaagttgagaaatgtaccggcttccatcctgtcaccatgacttcctcacatggtctgcttagcccttctctgctccttattcccagtgttccatttgaaccagtgatccatgtcctgaaaaatgctcaatctcagctaacattccatgtttcagaagcattcaggcactgccaggcttgcagtctcccagatgttgcatccctgaaacatctcaacaacctgaatgtcccaacccagacaatggcccaggtctctcaacttcatcagtgtggcttctatgagcccagatcaccacctgaacgttctgtctgtggcacattcttaaatatttccatcagcccatctcaacaatatatgtcctataaatggaccatccttgaca

SEQ ID NO: 8 - protein, 277 aa, Homo sapiens
MWPLALVIASLTLALSGGVSQESSKVLNTNGTSGFLPGGYTCFPHSQPWQAALLVQGRLLCGGVLVHPKWVLTAAHCLKEGLKVYLGKHALGRVEAGEQVREVVHSIPHPEYRRSPTHLNHDHDIMLLELQSPVQLTGYIQTLPLSHNNRLTPGTTCRVSGWGTTTSPQVNYPKTLQCANIQLRSDEECRQVYPGKITDNMLCAGTKEGGKDSCEGDSGGPLVCNRTLYGIVSWGDFPCGQPDRPGVYTRVSRYVLWIRETIRKYETQQQKWLKGPQ

SEQ ID NO: 9 - DNA, 25 bp, Artificial Sequence
  Description of Artificial Sequence Synthetic oligonucleotide
gctctagaatggctacagcaagacc

SEQ ID NO: 10 - DNA, 26 bp, Artificial Sequence
  Description of Artificial Sequence Synthetic oligonucleotide
tatccggagtctgaaaggagtgtcag